(19) United States
(12) Patent Application Publication
(10) Pub. No.: US 2012/0201779 A1
(43) Pub. Date: Aug. 9, 2012

(54) ANTIHISTAMINE AND ANTIHISTAMINE-LIKE NASAL APPLICATION, PRODUCTS

(75) Inventor: Ashok Wahi, Hillsborough, NJ (US)

(73) Assignee: TRUTEK CORPORATION, Hillsborough, NJ (US)

(21) Appl. No.: 13/446,134

(22) Filed: Apr. 13, 2012

Related U.S. Application Data
(62) Division of application No. 12/466,382, filed on May 14, 2009.
(60) Provisional application No. 61/091,887, filed on Aug. 26, 2008.

(51) Int. Cl. A61K 31/717; A61K 31/785; A61P 37/08; A61K 31/787; A61P 11/02

(52) U.S. Cl. 424/78.35; 514/57

(57) ABSTRACT

A method of and product for reducing the undesirable effects of allergic rhinitis by applying a formulation to a person's nasal region or nostrils. The formulation creates a barrier that prevents airborne allergens from contacting nasal mucous membranes, and at the same time, electrostatically repels or attracts and captures the particulate allergens, and changes their shapes so as to mitigate the effects of allergic rhinitis. The formulation may be made more effective by the addition of an anti-histamine compound.

### CROSS REFERENCE TO RELATED APPLICATIONS

This Present US patent application is a division of pending U.S. patent application Ser. No. 12/466,382 filed on May 14, 2009, which in turn is the non-provisional counterpart of pending U.S. Provisional Patent Application Ser. No. 61/091,887 filed on Aug. 28, 2008. Both U.S. patent application Ser. Nos. 12/466,382 and 61/091,887 are incorporated by reference in their entirety into the Present Application.

### FIELD OF THE INVENTION

The Present Invention relates to the field of protective compositions that work against assault by various airborne allergens that gain entry into the body through the airway and/or nasal mucosa. Typically, these allergens comprise, among other things, pollen, dust mite, mold, and animal dander. The Present Invention also relates to products that were heretofore developed for restricting the flow of airborne contaminants into the nasal passages by creating an electrostatic field in an area near or about the nasal passages. This reduced the inflow of airborne contaminants to the nasal passages by capturing the contaminants and keeping them from entering the body and stimulating an allergic response. The Present Invention also relates to formulations that have an antihistamine or an antihistamine-like effect on the body to remediate allergic reactions due to these airborne allergens.

### BACKGROUND OF THE INVENTION

Many individuals suffer from spring and fall allergies to airborne allergens. The main contaminant during the spring is tree pollen. During the fall season, the main contaminant is ragweed pollen. However, many individuals suffer allergic reactions all year round. Many of these reactions are to dust, dust mites, molds, and mildew. During allergy season, many sufferers are forced to breathe through their mouths due to their nasal passages being blocked. However, for most people, the nasal passages and nasal mucosa serve as the main entry points for most of these allergens. Breathing through one's nose is desirable, since the nasal passages have natural filters for airborne particulates, thereby preventing them from entering the lower respiratory system. The immune system's mechanism for dealing with allergens is for cells present in the nasal mucosa to produce chemical mediators (histamines are one example of many produced) within fifteen minutes after the allergens come in contact with cells present in the nasal mucosa, eyes, and lungs, etc. This occurs because the immune system perceives that the foreign airborne contaminants, particulate and/or objects that have entered the body may be harmful, and the allergic reaction thus triggered is designed to eject them from the body or negate their effect. Unfortunately, in most cases this allergic reaction does more harm than good. In a mild allergic reaction, substances such as histamines cause sniffling, sneezing, coughing, itchy throat, and itchy eyes. In some instances, the allergic reaction can be severe so as to cause urticaria (hives) and even anaphylactic shock. The term ‘histamine’ is used here in a broader sense so as to include several different types of mediators, many of which play a role in creating an allergic reaction at different stages. Therefore, the approved available alleviating remedies act differently on different mediators responsible for causing an allergic response. When the airborne allergens contact the nasal membranes, a person finds it difficult to breath through his nose.

Histamine release within the body is an over exaggerated immune response when an allergen enters the body. When the allergen touches the mucous membrane in the nasal passages, the immune system perceives an attack by a harmful substance. When histamines are released into the bloodstream, a person develops allergic rhinitis manifesting in a “runny” or “itchy” nose, sneezing, “watery” eyes, itchy throat, etc. This could happen immediately within the first 15 minutes or 4 to 8 hours later. Many prescription and over-the-counter anti-histamine medications are designed to suppress histamine and other mediator production in the body. One can also take nasal steroids or immunotherapy injections designed to reduce and modify the immune response to the allergen to lessen or eliminate the allergic reaction. Watery eyes and itchy throat may follow the reaction to the allergens in the nose, which is the primary trigger point. If the allergens in the nose can be prevented from contacting the mucous membranes, the allergic reaction may be greatly reduced.

Anti-histamines can work in two ways. In the first way, the anti-histamine medication is taken orally to provide systemic relief. Many such anti-histamines may make their users drowsy, and this is quite undesirable. Anti-histamine drugs exist that do not produce drowsiness, but many of them have other undesirable side effects. In the second way, the anti-histamine medication works topically. These are anti-histamine nasal sprays and eye drops. Here, the side effects are minimal. Although these nasal sprays work topically on the nose, they have some systemic absorption. Here, the suppression of histamine production begins locally, but it actually works both topically and systemically. Furthermore, even though the histamine reaction is suppressed, the airborne allergens continue to present themselves to the immune system, mainly through the nose.

Patents such as U.S. Pat. No. 5,468,488, U.S. Pat. No. 5,674,481, and U.S. Pat. No. 6,844,005 describe electrostatically charged compositions that may be applied externally near the nostril and attract oppositely charged materials that would otherwise be inhaled. Those compositions create an electrostatic field that helps to filter out oppositely charged materials.

It would be desirable if a formulation were to exist that, when applied externally as a cream, ointment, lotion, gel, or other topical formulation to the nasal area or when sprayed as a liquid into the nostrils, would capture allergens, prevent them from contacting the mucous membranes, and reducing the allergic reaction. It would also be desirable to include an antihistamine agent or an antihistamine-like product, to lessen the allergic reaction. A topical nasal decongestant may also be included. Such products are commonly known and used for treatment of allergies, and many of these products are currently sold on the market. One such product is sold with the proprietary label of Anthistine, and is used for itchy eyes. Other products that are antihistamine-like are as Nasonex (mometasone furoate monohydrate—a corticosteroid) and Afrin Nasal Spray.

### OBJECTS OF THE INVENTION

It is therefore an object of the invention to provide a formulation that can be readily applied to the exterior region around the nostril and/or slightly inside the edge of the nostril compositions capable of electrostatically attracting airborne allergens, capturing them, and rendering them harmless.
It is another object of the invention to provide a formulation that can be sprayed into the nose capable of electrostatically attracting airborne allergens, capturing them, and rendering them harmless.
It is a further object of the invention to provide a spray, cream, ointment, lotion, gel, or other topically applied formulation as outlined above which will have the additional property of insulating the nasal mucosa from contact with the captured allergens.
It is yet another object of the invention to add anti-histamine medications to the above formulations to locally suppress the formation of histamines.
Yet other objects of the invention will be apparent to those of ordinary skill once having benefit of the instant disclosure. In all of the foregoing objects, the deficiencies of the previously mentioned prior art are overcome by the teachings of this invention.

### SUMMARY OF THE INVENTION

These and other objects of the invention are unexpectedly achieved by method of providing formulations having at least one polymeric quaternary compound in an aqueous or non-aqueous based formulation, which when applied to a surface, forms a barrier and creates an electrostatic field such that oppositely charged airborne allergens in the vicinity of the surface are electrostatically attracted, and captured. The barrier prevents the allergens from having contact with the nasal mucosa, thereby lessening their harmful effects. Combined with known antihistamine medications, allergic reactions can be alleviated, with or without synergy from two modes of action.

### DETAILED DESCRIPTION OF THE INVENTION

The Present Invention comprises a methodology and also product formulations. The formulations are either included within gels that are applied within the nostrils and around the nasal area or within liquids that are sprayed into the nostrils. Upon coming in contact with the inside surface of the nostrils, a film or barrier insulates the mucous membranes. The formulations contain a commonly used cationic agent that creates an electrostatic field. This has the effect of attracting the allergens, which are oppositely charged. The barrier is capable of then capturing the invading allergens. This reduces the allergic rhinitis reaction including the release of histamines and other mediators without utilizing a topical or systemic drug medication. The effect of this is a reduced allergic response. For example, if a reaction begins to occur in an individual allergic to ragweed pollen when the pollen count reaches a threshold, upon application of the formulation, the reaction might not begin to occur until the pollen count is significantly higher. Preliminary observations of test subjects for a formulation of the Present Invention indicate that the total and the specific IgE protein are reduced. Within a short time following application, these subjects report that their symptoms of allergic rhinitis virtually disappear or are reduced. First, the eyes and throat stop itching, then the “sniffles” stop, and finally the subjects are able to breathe comfortably through their noses. In most, but not all, subjects, the symptoms disappear in the reverse order in which they appeared at the inception of the allergic reaction.

Previous products either attracted or repelled electrostatically charged airborne particles by application to a region proximate to the nostrils. Those particles that were repelled away from the applied product never entered the nostrils. Those particles that were attracted to the applied product also never entered the nostrils because they were captured and trapped within the product itself. Therefore, the number of particles that would enter the nostrils was greatly reduced. At some point, the previous products were removed by wiping, and then reapplied when desired. The product of the Present Invention operates differently from these previous products. In addition to its application to the vicinity of the nostrils, it is also meant to be applied inside the nasal passages. The product of the Present Invention both attracts airborne allergens and creates a barrier between the air and the mucous membranes in the nostrils. It can be in the form of a gel or a spray. Not only are the airborne allergens prevented from contacting the mucous membranes, but they are also rendered harmless by contact with the product itself. This effect is synergistic.

To accomplish the Present Invention, a formulation having at least one cationic agent known in the art such as a polyquaternary ammonium compound is prepared, such compounds, alone or together capable of creating an electrostatic field on and around a surface to which it is applied, including surfaces such as skin, textile (woven and non-woven), and hard surfaces, such as floors, walls, wood, metal, plastic, etc. The formulation is generally aqueous based, but may include non-aqueous solvents used which are compatible with the other formulation components and the application surface to which it is applied. Preferably, the formulation is an aqueous formulation. In addition, the composition may contain, but is not required to contain various thickeners, gellants, fragrances, colorants, emollients, humectants, and generally other suitable components that are compatible with the end use application and the other components of the formulations.

Most airborne allergen particulates, such as pollen, dust, etc., though small, are not as minute as most microorganisms. Although some are microscopic, many can be observed with the naked eye. The shapes of these particulates comprise fibrous extensions that enable them to stick to mucous membranes. In many respects, it resembles a lint ball or a ball of cotton candy. These extensions are what cause the allergic reaction to commence. However, once the formulation of the Present Invention is applied to the nostrils and the surrounding nasal area, the instant that the allergen particulates touch the barrier, their shapes change and the extensions flatten out. In this mode, these particulates are rendered less harmful. They cannot penetrate the mucosa to cause an allergic reaction. Even should they be dislodged after initial capture, they become trapped in the nasal hairs, and are rendered ineffective. It is true that some particulates will still remain active (perhaps in the bronchial tubes, or lungs). However, by capturing most of these particles and rendering them ineffective, the trigger threshold of individual subjects increases.

The effectiveness of the product may be improved by combining it with an antihistamine compound, such as one known in the art. However, the combination of the electrostatic barrier along with localized application of antihistamines may exhibit a three-step synergistic effect. First, most allergen particulates are prevented from coming in contact with the nasal mucosa by the barrier. Second, the particulates are captured and their shapes are changed, thereby rendering them ineffective. These two elements reduce the severity of the allergic reaction. Finally, because the allergic reaction is milder, the anti-histamines are more effective in remediating or eliminating the effects of the reaction.

A formulation of the invention comprises:
water,
at least one quaternary thickener,
a preservative,
an emulsifier,
a biocidic agent, and
a neutralizing agent added to adjust and achieve a pH in the range of 5.0 to 6.8.

In an exemplary embodiment of such a formulation, the amount of water may range from 60% to 90% by weight; quaternary thickener (at least one must be present)—0.5% to 5.0% by weight; preservative—0.1% to 1.0% by weight; emulsifier—0.1% to 3.0% by weight; and biocidic agent—0.1% to 1.0% by weight.

In an exemplary embodiment of such a formulation, a quaternary thickener may comprise, without limitation, at least one of the following:
Polyquaternium-10;
Polyquaternium-22;
Polyquaternium-67;
Polyquaternium-91.

In an exemplary embodiment of such a formulation, an emulsifier may comprise, without limitation, at least one of the following:
cetyl alcohol;
cetearyl alcohol;
glyceryl stearate;
Ceteareth-20.

In an exemplary embodiment of such a formulation, an emollient may comprise, without limitation, at least one of the following:
C 10-30 Cholesterol/Lanosterol Esters;
ethylhexyl palmitate;
hydrogenated Polyisobutene.

In an exemplary embodiment of such a formulation, a preservative may comprise, without limitation, at least one of the following:
phenoxyethanol;
methylparaben;
butylparaben;
ethylparaben;
propylparaben;
isobutylparaben.

Examples of typical formulations would comprise the following compositions:

TABLE 1

| Ingredient | Percent Range | Function |
|---|---|---|
| Water | 80%-90% | Solvent, Moisturizer |
| Polyquaternium-10 | 2%-5% | Conditioner, Quaternary, |
| | | Thickener |
| Polyquaternium-67 | 1%-2% | Conditioner, Quaternary, |
| | | Thickener |
| Phenoxyethanol, | 1% | Preservative |
| Methylparaben, | | |
| Butylparaben, | | |
| Ethylparaben, | | |
| Propylparaben, | | |
| Isobutylparaben | | |
| Phenoxyethanol | 0.2%-0.3% | Preservative, Masking |
| | | Agent |
| Polyquaternium-22 | 1%-2% | Conditioner, Quaternary |
| Cetronium Chloride | 2% | Conditioner, Quaternary |
| C10-30 Cholesterol/ | 0.2%-0.3% | Emollient |
| Lanosterol Esters | | |
| Cetyl Alcohol | 2% | Thickener, Auxiliary |
| | | Emulsifier |
| Cetearyl Alcohol, | 2%-3% | Emulsifier |
| Glyceryl Stearate, | | |
| PEG-40 Stearate, | | |
| Ceteareth-20 | | |
| Benzalkonium Chloride | 0.5% | Biocide, Conditioner, |
| | | Quaternary |
| Hydroxypropyl Trimonium | 0.5% | Conditioner, Quaternary |
| Hydrolized Silk | | |
| Sodium Hydroxide | 0.025% | Neutralizing Agent |

The formulation for a first embodiment of the invention is shown in Table 1. The functionality of each ingredient is also shown the table. As can be seen, the composition is comprised mainly of water. The other ingredients constitute between 10%-30% by weight. The pH of the composition ranges from 6.1 to 6.5, and the viscosity varies from 50,000-110,000 cps. The specific gravity ranges from 0.96-1.02.

Other typical formulations follow:

TABLE 2

| Ingredient | Percent | Functionality |
|---|---|---|
| Water | 80%-90% | Solvent, moisturizer |
| Quaternium-91, | 1%-4% | Conditioner |
| Cetrimonium Methosulfate, | | |
| Cetearyl Alcohol | | |
| Stearyl Alcohol | 2% | Thickener |
| Cetyl Alcohol | 0.5% | Thickener |
| C10-30 Cholesterol, | 1% | Emollient |
| Lanosterol Esters | | |
| Ethylhexyl Palmitate | 3%-5% | Emollient |
| Glyceryl Stearate, | 1%-3% | Emulsifier |
| PEG-100 Stearate | | |

TABLE 2 shows the formulation for a second embodiment of the invention. As is evident from TABLE 2, Quaternium-91 is used in this embodiment instead of Polyquaternium-10, Polyquaternium-22, and Polyquaternium-67 from the first embodiment.

TABLE 3

| Ingredient | Percent | Functionality |
|---|---|---|
| Water | 80%-90% | Solvent, moisturizer |
| Dipropylene Glycol | 2%-4% | Emollient |
| Acetamide MEA | 1% | Humectant |
| Gluconolactone, | 1% | Preservative |
| Sodium Benzoate | | |
| Quaternium-91, | 1%-4% | Conditioner |
| Cetrimonium Methosulfate, | | |
| Cetearyl Alcohol | | |
| Cetearyl Alcohol, | 1%-4% | Thickener |
| Cocoa Glucoside | | |
| Cetyl Alcohol | 0.1%-1.5% | Thickener |
| C10-30 Cholesterol, | 0.5%-1.5% | Emollient |
| Lanosterol Esters | | |
| Ethylhexyl Palmitate | 2%-6% | Emollient |
| Glyceryl Stearate, | 1%-4% | Emulsifier |
| PEG-100 Stearate | | |
| Hydroxypropyl | .′0.5% | Skin Conditioner |
| Trimonium Hydrolyzed | | |
| Oat Protein | | |
| Water, | 1% | Skin Conditioner |
| Hydrolyzed Algin | | |

TABLE 3 represents the formulation for a third embodiment of the Present Invention. Note that the Quaternium-91 component is present in the same percentage in both the second and third embodiments.

TABLE 4

| Ingredient | Percent | Functionality |
|---|---|---|
| Water | 75%-85% | Solvent, Moisturizer, Surfactant |
| Gluconolactone, | 1% | Preservative |
| Sodium Benzoate | | |
| Quaternium-91, | 1%-4% | Conditioner |
| Cetrimonium | | |
| Methosulfate, | | |
| Cetearyl Alcohol | | |
| Stearyl Alcohol | 1%-3% | Thickener |
| Cetyl Alcohol | 0.1%-1.5% | Thickener |
| C10-30 Cholesterol | 1%-3% | Emollient |
| Lanosterol Esters | | |
| Ethylhexyl Palmitate | 2%-6% | Emollient |
| Glyceryl Stearate, | 1%-4% | Emulsifier |
| PEG-100 Stearate | | |
| Polyquaternium - 22 | 0.5%-3% | Conditioner |
| Ethoxyethanol | 0.5% | Preservative |
| Sodium Hydroxide | 3% | Neutralizing agent |

TABLE 4 represents the formulation for a fourth embodiment of the Present Invention. Note the use of both Quaternium-91 and Polyquaternium-22.

TABLE 5

| Ingredient | Percent | Functionality |
|---|---|---|
| Water | 70%-80% | Solvent, moisturizer |
| Gluconolactone, | 1% | Preservative |
| Sodium Benzoate | | |
| Quaternium-91, | 1%-4% | Conditioner |
| Cetrimonium Methosulfate, | | |
| Cetearyl Alcohol | | |
| Stearyl Alcohol | 1%-3% | Thickener |
| Cetyl Alcohol | 0.1%-1.5% | Thickener |
| C10-30 Cholesterol | 1%-3% | Emollient |
| Lanosterol Esters | | |
| Hydrogenated Polyisobutene | 3%-6% | Emollient |
| Glyceryl Stearate, | 1%-4% | Emulsifier |
| PEG-100 Stearate | | |
| Polyquaternium - 22 | 2%-5% | Conditioner |
| Phenoxyethanol | 0.5% | Preservative |
| Hydroxypropyl Trimonium | 0.5% | Conditioner |
| Hydrolyzed Silk | | |
| Hydrolyzed Milk Protein | 0.25% | Conditioner |
| Sodium Hydroxide | 3%-4% | Neutralizing agent |

TABLE 5 represents the formulation for a fifth embodiment of the Present Invention.

TABLE 6

| Ingredient | Percent | Functionality |
|---|---|---|
| Water | 75%-85% | Solvent, moisturizer |
| Polyquaternium - 67 | 0.5%-2% | Conditioner/Quat |
| Phenoxyethanol, | 1% | Preservative |
| Methyloaraben, | | |
| Butylparben, | | |
| Ethylparaben, | | |
| Propylparaben, | | |
| Isobutylpareben | | |
| Polyquaternium 22 | 1%-4% | Conditioner/Quat |
| Cetyl Trimethyl Ammonium | 1%-3% | Conditioner/Quat |
| Chloride | | |
| Stearyl Alcohol | 1%-2% | Thickener |
| Cetyl Alcohol | 1%-2% | Thickener |
| C10-30 Cholesterol | 0.5%-2% | Emollient |
| Lanosterol Esters | | |
| Hydrogenated Polyisobutene | 3%-6% | Emollient |
| Glyceryl Stearate, | 1%-4% | Emulsifier |
| PEG-100 Stearate | | |
| Hydroxypropyl Trimonium | 0.5% | Conditioner/Quat |
| Hydrolyzed Silk | | |
| Benzylknonium Chloride | 1%-3% | Conditioner/Quat |
| Sodium Hydroxide | 1% | Neutralizing agent |

TABLE 6 represents the formulation for a sixth embodiment of the Present Invention. Note the inclusion of Polyquaternium-67 and Polyquaternium-22 as well as the inclusion of Benzylknonium Chloride.

All of the formulations described in TABLE 1-6 representing the six embodiments of the Present Invention operate in the manner that was disclosed herein. The same results may also be achieved by varying the percentages for the active and inactive ingredients. Varying the percentages for the active ingredients affects the potency of the formulation. Varying the percentages for the inactive ingredients affects the consistency of the formulation. The desired results may be achieved by varying the ingredients and their amounts by those skilled in the art without undue experimentation.

1. A formulation to be applied to a person's nasal region or nostrils which reduces the undesirable effects of allergic rhinitis, said formulation comprising at least one cationic agent, and wherein said formulation, once applied,
a) forms a barrier that prevents airborne allergens from contacting nasal mucous membranes; and
b) creates an electrostatic field that attracts airborne allergens in the vicinity of the nasal region or nostrils and captures them;
and further comprising a neutralizing agent to adjust and achieve a pH in the range of 5.0 to 6.8.

2. The formulation of claim 1 wherein the at least one cationic agent is a polymeric quaternary ammonium compound.

3. The formulation of claim 1 further comprising a liquid to facilitate spraying into the person's nostrils.

4. The formulation of claim 3 wherein the liquid is water.

5. The formulation of claim 1 further comprising a gel or cream to facilitate manual application of the formulation around the person's nasal region and nostrils.

6. The formulation of claim 1 further comprising an antihistamine compound.

7. The formulation of claim 1 further comprising a topical nasal decongestant.

8. The formulation of claim 1 further comprising a corticosteroid.

9. A formulation to be applied to a person's nasal region or nostrils which reduces the undesirable effects of allergic rhinitis, said formulation comprising by weight:
between 2% and 5% Polyquaternium-10;
between 1% and 2% Polyquaternium-67; and
between 1% and 2% Polyquaternium-22.

10. The formulation of claim 9 further comprising Cetronium Chloride, and Benzalkonium Chloride.

11. The formulation of claim 9 further comprising at least one emulsifier.

12. The formulation of claim 11 wherein the emulsifier is Cetyl Alcohol.

13. The formulation of claim 9 further comprising at least one preservative.

14. The formulation of claim 9 further comprising at least one emollient.

15. The formulation of claim 9 further comprising at least one masking agent.

16. The formulation of claim 9 further comprising at least one thickener.

17. A formulation to be applied to a person's nasal region or nostrils which reduces the undesirable effects of allergic rhinitis, said formulation comprising at least one substance taken from the group consisting of:
Quaternium-91;
Polyquaternium-10;
Polyquaternium-22;
Polyquaternium-67; and
Benzalkonium Chloride,
wherein the pH of the formulation is in the range of 5.0 to 6.8.

18. The formulation of claim 17 further comprising at least one emulsifier.

19. The formulation of claim 17 further comprising at least one preservative.

20. The formulation of claim 17 further comprising at least one emollient.

21. The formulation of claim 17 further comprising at least one masking agent.

22. The formulation of claim 17 further comprising at least one thickener.

23. A formulation to be applied to a person's nasal region or nostrils which reduces the undesirable effects of allergic rhinitis, said formulation comprising:
a) water;
b) at least one quaternary thickener;
c) a preservative;
d) an emulsifier;
e) a biocidic agent; and
f) a neutralizing agent added to adjust and achieve a pH in the range of 5.0 to 6.8.

24. The formulation of claim 23 wherein:
a) the amount of water ranges from 60% to 90% by weight;
b) the amount of quaternary thickener ranges from 0.5% to 5.0% by weight;
c) the amount of preservative ranges from 0.1% to 1.0% by weight;
d) the amount of emulsifier ranges from 0.1% to 3.0% by weight; and
e) the amount of biocidic agent ranges from 0.1% to 1.0% by weight.

25. The formulation of claim 23 wherein the at least one quaternary thickener is taken from the group consisting of:
Polyquaternium-10,
Polyquaternium-22,
Polyquaternium-67, and
Polyquaternium-91.

26. The formulation of claim 23 wherein the emulsifier is taken from the group consisting of:
cetyl alcohol,
cetearyl alcohol,
glyceryl stearate, and
Ceteareth-20.

27. The formulation of claim 23 wherein the emollient is taken from the group consisting of:
C 10-30 Cholesterol/Lanosterol Esters,
ethylhexyl palmitate, and
hydrogenated Polyisobutene.

28. The formulation of claim 23 wherein the preservative is taken from the group consisting of:
phenoxyethanol,
methylparaben,
butylparaben,
ethylparaben,
propylparaben, and
isobutylparaben.
